(19) United States
(12) Patent Application Publication
(10) Pub. No.: US 2006/0084101 A1
(43) Pub. Date: Apr. 20, 2006

(54) Two-color chemiluminescent microarray system

(75) Inventor: Tom C. Xu, Castro Valley, CA (US)

Correspondence Address: HELLER EHRMAN LLP, 275 MIDDLEFIELD ROAD, MENLO PARK, CA, 94025-3506, US

(73) Assignee: Applera Corporation, Foster City, CA (US)

(21) Appl. No.: 11/241,127

(22) Filed: Sep. 29, 2005

Related U.S. Application Data
(60) Provisional application No. 60/617,292, filed on Oct. 7, 2004.

(51) Int. Cl. C12Q 1/68; G01N 33/53

(52) U.S. Cl. 435/6; 435/7.5

(57) ABSTRACT

Methods, apparatus and systems for detecting target molecules in samples are provided.

### CROSS-REFERENCE TO RELATED APPLICATIONS

This application claims priority under 35 U.S.C. § 119(e) to U.S. Provisional Application Ser. No. 60/617,292, filed Oct. 7, 2004, the entire contents of which are hereby incorporated herein by reference.

### BACKGROUND

### Field

This invention pertains to assays, systems and methods for detecting target molecules. In particular, the invention pertains to the use of chemiluminescent labels in the detection and identification of target molecules.

Methods and systems for detecting target molecules in a sample and for identifying target molecules are provided herein. For example, methods and systems for detecting two different target molecules in a sample at the same time are provided. Targets may bind to a probe or probes to form a target-probe conjugate. Target molecules may be, for example, DNA (e.g., cDNA molecules), RNA, reverse transcriptase products, polymerase chain reaction (PCR) products, ligation chain reaction (LCR) products, peptides, proteins or other molecules. Target molecules may be detected at the same time by detecting fluorescent signals from one, two or more of a plurality of chemiluminescent markers reactive with a single enzyme substrate. Chemiluminescent markers may be, for example, luciferases. Compositions, including surfaces adorned with probes having such chemiluminescent target-probe conjugates, are also provided.

The target molecules may hybridize with, or otherwise bind to, probes, the probes and targets being effective to form a probe-target conjugate when in contact with each other. Target molecules have, or are modified to have, a “tag” that is complementary to a molecule (an “anti-tag”) that is effective to combine with the tag. The chemiluminescent markers (e.g., luciferases) are linked to anti-tags complementary to tags attached to the target molecules. Tags and anti-tags are effective to combine together so as to link chemiluminescent markers with target molecules. In some embodiments, at least two target molecules can be detected, each linked to a different tag molecule, each different tag being effective to combine with a corresponding complementary anti-tag molecule that is linked to a corresponding chemiluminescent marker selected from at least two different chemiluminescent markers.

Target molecules bound to probes (target-probe conjugates) may be detected by detection of light emitted by the chemiluminescent markers. A combination or combinations of target molecules with probe molecules to form target-probe conjugates may thus be detected by contacting the tagged target molecules with the chemiluminescent markers coupled to molecules or moieties complementary to the tags (anti-tag molecules), and detecting chemiluminescent light emission. Probes may be attached to a solid support, such as a support surface (e.g., a microarray surface), and binding may be detected by detection of chemiluminescent target-probe conjugates on the support. Target molecules may be identified by detection of chemiluminescence at a particular location on a microarray surface, indicating that a target has bound to the probe attached to the substrate at that location. In some embodiments, a particular chemiluminescent marker corresponds with a particular target molecule, and detection of that particular chemiluminescent signal at an identified location of a microarray is effective to identify the particular target molecule.

In one aspect, a two-color target molecule assay system is provided that utilizes two chemiluminescent molecules having different light emitting properties, and methods for detecting different target molecules at the same time using two chemiluminescent enzymes having different light emitting properties. The chemiluminescent molecules are preferably chemiluminescent enzymes having similar or identical enzyme substrates and having similar pH and buffer requirements, compatible kinetics, and high sensitivity under similar or identical conditions.

In some methods disclosed herein, target molecules or target molecule copies are coupled with first and second tags (e.g., tagged cDNAs are produced from a sample of target material) and contacted with a solution including a first luciferase conjugated with a first molecule complementary to the first tag (a first anti-tag) and a second luciferase conjugated with a second molecule complementary to the second tag (a second anti-tag). Binding of the complementary tags and anti-tags is effective to link the luciferases with the target molecules or target molecule copies, producing a labeled target by linking the target with a chemiluminescent label (i.e., a luciferase). Addition of luciferin, an enzyme substrate that is compatible with both luciferases, is effective to catalyze light-emitting reactions between the luciferases and the substrate, providing light with dual intensity peaks centered around a first λₘₐₓ and a second λₘₐₓ characteristic of the first and second luciferase, respectively. Measurement of light emission allows detection of different target molecules at the same time without need for sequential reactions.

In another aspect of the methods and apparatus disclosed herein, support surfaces comprising a probe, or preferably a plurality of probes are provided. The array may be a microarray having a plurality of probes, preferably a plurality of first probes and a plurality of second probes, or may be a microtitre plate. For example, a first probe may be configured to bind a first target molecule coupled to a first luciferase having a first λₘₐₓ (e.g., via a tag bound to an anti-tag) and a second probe configured to bind a second target molecule coupled to a second luciferase having a second λₘₐₓ (e.g., via a tag bound to an anti-tag).

In one aspect of the methods disclosed herein, a method for detecting labeled target molecules present in a sample is provided. A first target molecule tagged with a first tag and a second target molecule tagged with a second tag are contacted with luciferase molecules having complementary anti-tag moieties. A luciferase may emit electromagnetic radiation at one wavelength, at a number of wavelengths or over a range of wavelengths. The emission wavelength or wavelengths preferably may include electromagnetic radiation at a visible wavelength or visible wavelengths. Emission of electromagnetic radiation is termed “light emission” regardless of the wavelength or wavelengths of that radiation. Different luciferase enzymes may have different chemiluminescent emission peaks; in addition, over a range of emisions wavelengths, a single luciferase enzyme may have multiple local emission peaks. Where the intensity of the light emission varies between wavelengths or over a range of wavelengths, the wavelength of greatest intensity is termed the λₘₐₓ.

The first luciferase has a first λₘₐₓ, and the second luciferase has a second λₘₐₓ that is different than the first λₘₐₓ. The first tag combines with the anti-tag coupled to the first luciferase, and the second tag combines with the second antitag coupled to the second luciferase, forming labeled target molecules. The first target is labeled with a first luciferase, and the second target is labeled with the second luciferase. The methods further include providing a luciferase substrate, effective to induce light emission from both the first luciferase and from the second luciferase; and detecting light that is emitted from the luciferases. Light emission near both the first λₘₐₓ and the second λₘₐₓ may be detected at the same time.

For example, the first luciferase may have a first λₘₐₓ of less than about 600 nm, and the second luciferase may have a second λₘₐₓ greater than about 600 nm. In some embodiments, the first λₘₐₓ is between about 540 nm and about 590 nm, and the second λₘₐₓ is between about 600 nm and about 650 nm. More preferably, the first λₘₐₓ is between about 550 nm and about 560 nm, and the second λₘₐₓ is between about 610 nm and about 620 nm. In other embodiments, the first λₘₐₓ is about 556 nm and the second about λₘₐₓ 618 nm.

In some embodiments, the first target molecule is tagged with a biotin tag, the second target molecule is tagged with a digoxigenin tag. In such an embodiment, the first anti-tag complex includes streptavidin coupled with a first luciferase having a first λₘₐₓ, and the second anti-tag complex includes an anti-digoxigenin antibody coupled with a second luciferase having a second λₘₐₓ. Such a first anti-tag complex binds to the first target molecule via streptavidin-biotin binding, and the second anti-tag complex binds to the second labeled target molecule via anti-digoxigenin antibody binding to digoxigenin.

Further methods include providing a probe molecule that is complementary to a target molecule, or preferably providing a plurality of probe molecules complementary to a target molecule or to multiple target molecules. In preferred methods, at least one probe molecule included in a plurality of probe molecules is complementary to each of the target molecules. Thus, for example, where there are two different target molecules, there may be at least two different probe molecules, one complementary to one target molecule, the other complementary to the other target molecule.

A probe molecule may be bound to a solid support. The solid support may be a surface, such as a substantially flat surface. In preferred methods, an array of probe molecules may be attached to a solid surface, which may preferably be a substantially flat surface. Such probe molecules and such solid surface may comprise a microarray of probe molecules arrayed on the surface, may comprise a microtitre plate having probe molecules attached to a surface in a well, or other arrangement. The novel methods, devices and systems thus may provide two-color assay systems and novel microarrays useful in such systems, and methods for using them.

Labeled support surfaces, including microarrays, wells and microtitre plates having wells with well surfaces, and are also provided. Such surfaces may include a plurality of probes attached to a solid surface, a labeled first target molecule hybridized (or hybridizable) to at least one probe, a first conjugate capable of binding to the first labeled target molecule, a second labeled target molecule hybridized (or hybridizable) to a probe, and a second conjugate capable of binding to the second labeled target molecule. For example, a labeled first target molecule may be a biotinylated target molecule and a first conjugate may, for example, include streptavidin conjugated with a first luciferase having a first λₘₐₓ. A second labeled target molecule may be, for example, a digoxigenin labeled target molecule, and a second conjugate may include an anti-digoxigenin antibody conjugated with a second luciferase having a second λₘₐₓ.

An advantage of the present methods and systems is that a single enzyme substrate, and one set of substrate conditions, may be used to provide two or more different chemiluminescent light signals under the same conditions and which may be measured at the same time. Thus, in some methods, luciferin is provided at a concentration of between about 0.5 mM and about 5 mM in a substrate solution comprising ionized magnesium (Mg⁺⁺) at a concentration of between about 2 mM and about 10 mM and ATP at a concentration of between about 1 mM and about 5 mM at a pH of between about 7.5 and about 8.5. The same substrate solution is used to generate light emission from both luciferases. In other embodiments, substrate solutions include ionized magnesium (Mg⁺⁺) at a concentration of between about 4 mM and about 6 mM and ATP at a concentration of between about 1 mM and about 3 mM.

Systems, devices and methods disclosed herein allow the simultaneous detection of multiple target molecules in a single assay, providing a two-color homogeneous assay in which only one enzyme substrate is required, and providing advantages over prior systems, devices and methods. The novel systems, devices and methods utilize marker molecules emitting light having different λₘₐₓ values, providing marker signals that are easily detected and readily distinguishable. By using luciferase enzymes having different chemiluminescent emission peaks, assay conditions can be optimized for both enzymes at once, providing a robust assay with extremely high sensitivity, utilizing enzymes with the highest known quantum yield available. Other features and advantages are described in the detailed description and examples presented below.

### BRIEF DESCRIPTION OF THE DRAWINGS

FIG. 1 depicts the chemical reaction mediated by firefly luciferase changing firefly luciferin to firefly oxyluciferin, leading to the emission of light.

FIG. 2 depicts a schematic representation of targets and probes forming target-probe conjugates, the probes being attached to a substrate.

FIG. 3 depicts a schematic representation of a microarray having probes attached to a substrate surface, the probes forming target-probe conjugates detectable by chemifluorescent light emitted by markers attached to anti-tags linked to tags attached to target molecules.

FIG. 4 depicts a series of steps in a method for detecting a target molecule.

FIG. 5 depicts a series of steps in a method for identifying a target molecule.

### DETAILED DESCRIPTION

A. Definitions

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Nucleotide” refers to a phosphate ester of a nucleoside, as a monomer unit or within a polynucleotide polymer. A polynucleotide, also termed a nucleic acid, may be composed entirely of deoxyribonucleotides, entirely of ribonucleotides, or chimeric mixtures thereof. Polynucleotides may be comprised of internucleotide, nucleobase and sugar analogs. Unless denoted otherwise, whenever a polynucleotide sequence is represented, it will be understood that the nucleotides are in 5′ to 3′ orientation from left to right and that “A” denotes deoxyadenosine, “C” denotes deoxycytidine, “G” denotes deoxyguanosine, and “T” denotes thymidine.

“Nucleotide analogs,” also termed nucleic acid analogs, include PNA, LNA, 2′-O-alkyl oligonucleotides, 3′ modified oligodeoxyribonucleotides, N3′-P5′ phosphoramidate (NP) oligomers, MGB-oligonucleotides (minor groove binder-linked oligonucleotides), phosphorothioate (PS) oligomers, C₁-C₄ alkylphosphonate oligomers, phosphoramidates, β-phosphodiester oligonucleotides, and α-phosphodiester oligonucleotides.

“Polynucleotides” may include nucleotides, nucleotide analogs, or mixtures of nucleotides and nucleotide analogs. Polynucleotides are not limited to any particular length of nucleotide sequence, as the term “polynucleotides” encompasses polymeric forms of nucleotides of any length, including oligonucleotides and nucleic acids. Polynucleotides that range in size from about 5 to about 40 monomeric units are typically referred to in the art as oligonucleotides. Polynucleotides that are several thousands or more monomeric nucleotide units in length are typically referred to as nucleic acids. Polynucleotides can be linear, branched linear, or circular molecules.

A molecule having a sequence of bases, such as a polynucleotide or polynucleotide analog, is termed a “nucleobase.” A sequence of bases is capable binding (e.g., hybridizing) to a complementary sequence of bases. As used herein, the expressions “sequence specificity” or “sequence specific” mean the hybridization of two or more polymeric nucleobase sequences by hydrogen bonding interactions that result from complementary base pairing.

As used herein, the terms “complementary” or “complementarity” are used in reference to antiparallel strands of nucleobases (i.e., a sequence of nucleobases) related by the Watson/Crick and Hoogsteen-type base-pairing rules. For example, the sequence 5′-AGTTC-3′ is complementary to the sequence 5′-GAACT-3′.

The term “hybridization” means the base-pairing interaction of one polynucleobase with another polynucleobase that results in formation of a duplex or other higher-ordered structure. The primary interaction is base specific, i.e., A/T and G/C, by Watson/Crick and Hoogsteen-type hydrogen bonding.

As used herein, “target”, “target polynucleotide”, “target sequence” and the like refer to a specific molecule (e.g., a polynucleotide or a polypeptide) sequence that is capable of attaching, linking, or binding to a a complementary molecule (a probe). For example, the target may be the subject of hybridization with a polynucleotide having a complementary base sequence. The nature of the target sequence is not limiting, and can be a polynucleotide, a peptide, protein, or other target molecule. A target polynucleotide may have any sequence, and be composed of, for example, DNA, RNA, substituted variants and analogs thereof, or combinations thereof. The target may also bind or link to, for example, an antibody. A target molecule for use with the present invention may be derived from any living or once living organism, including but not limited to prokaryote, eukaryote, plant, animal, and virus, as well as non-natural, synthetic and/or recombinant target sequences.

As used herein, the term “probe” refers to a nucleobase, antibody, or other molecule with affinity for a target. For example, a probe may be a polynucleotide that is capable of forming a duplex structure by complementary base pairing with a sequence of a target polynucleotide. A probe may also be, e.g., an antibody.

The combined probe and target, when linked, bound, or otherwise attached together, is termed a “probe-target conjugate” or “target-probe conjugate.”

In some embodiments, the probe is fixed to a solid support, such as in column, a chip, a bead, a flat surface, or other array format. A plurality of probes may be fixed to a solid support at known positions. A probe, and preferably a probe-target conjugate, is detected, visualized, measured and/or quantitated. A probe fixed to a solid support at a known position allows the determination of the location and identity of a target linked to the probe.

The term “solid support” refers to any solid phase material upon which an oligonucleotide is synthesized, attached or immobilized. Solid support encompasses terms such as “resin”, “solid phase”, “support” and “support surface.” The solid support may be any solid support, including a bead, gel, or other solid material. The solid support may be a substantially flat surface, such as, e.g., a glass microscope slide, including a flat surface with a well or wells. A solid support, such as a bead, may have a solid surface substantially excluding material from some or all of the interior volume of the solid support, or may have a porous or convoluted surface allowing access to some or all of the interior volume of the solid support.

The term λₘₐₓ refers to the wavelength of maximum intensity of light emitted by a chemiluminscent molecule. The λₘₐₓ of a chemiluminescent molecule is typically characteristic of that molecule under a given set of conditions; however, the λₘₐₓ of a particular chemiluminescent molecule may vary depending upon the pH of a solution in which the molecule is found, or the salt concentration, or other factor.

Detection of emitted light at a particular wavelength is typically performed by detecting the intensity of radiation emitted within a small range or “window” of wavelengths centered at that particular wavelength. Such a small range, or window, may include, for example, wavelengths a few nm above and below the particular “nominal” wavelength. Thus, a window around a λₘₐₓ may include, for example wavelengths within about 5 nm of the λₘₐₓ (e.g., from 595 nm to 605 nm where λₘₐₓ is 600 nm) or within about 1 nm of the λₘₐₓ (e.g., from 599 nm to 601 nm where λₘₐₓ is 600 nm). The strength of the detected signal is proportional to the size of the window; however, increasing window size may also increase noise, and decreases resolution.

As used herein, “support bound” means immobilized on or to a solid support. It is understood that immobilization can occur by any means, including for example; by covalent attachment, by electrostatic immobilization, by attachment through a ligand/ligand interaction, by contact or by depositing on the surface.

As used herein, the terms “array” or “microarray” indicate a predetermined spatial arrangement of probes (e.g., polynucleotides or antibodies) or targets present on a solid support and/or in an arrangement of vessels. Certain array formats are referred to as a “chip” or “biochip” (M. Schena, Ed. Microarray Biochip Technology, BioTechnique Books, Eaton Publishing, Natick, Mass. [2000]). An array can comprise a low-density number of addressable locations, e.g., 2 to about 12, medium-density, e.g., about a hundred or more locations, or a high-density number, e.g., a thousand or more. Typically, the array format is a geometrically-regular shape which allows for facilitated fabrication, handling, placement, stacking, reagent introduction, detection, and storage. The array may be configured in a row and column format, with regular spacing between each location. Alternatively, the locations may be bundled, mixed, or homogeneously blended for equalized treatment or sampling. An array may comprise a plurality of addressable locations configured so that each location is spatially addressable for high-throughput handling, robotic delivery, masking, or sampling of reagents. An array can also be configured to facilitate detection or quantitation by any particular means, including but not limited to, scanning by laser illumination, confocal or deflective light gathering, and chemical luminescence. In its broadest sense, “array” formats, as recited herein, include but are not limited to, arrays (i.e., an array of a multiplicity of chips), microchips, microarrays, a microarray assembled on a single chip, or any other similar format.

The term “sample” as used herein is used in its broadest sense. A “sample” is typically, but not exclusively, of biological origin, and can refer to any type of material obtained from animals or plants (e.g., any fluid or tissue), cultured cells or tissues, cultures of microorganisms (prokaryotic or eukaryotic), any fraction or products produced from a living (or once living) culture or cells, or synthetically produced or in vitro sample. A sample can be unpurified or purified. A purified sample can contain principally one component, e.g., total cellular RNA, total cellular mRNA, cDNA or cRNA, PCR products, or other molecules. In some embodiments, a sample can comprise material from a non-living source, such as synthetically produced nucleobase polymers or polypeptides.

As used herein, the term “in vitro” refers to an artificial environment and to processes or reactions that occur within an artificial environment. The term “in vivo” refers to the natural environment (e.g., in an animal or in a cell) and to processes or reactions that occur within a natural environment.

A “linking moiety” is a molecule or portion of a molecular capable of binding, linking, or otherwise attaching to a complementary molecule in a substantially specific manner. A “linking molecule” or “linker molecule” is a molecule having a linking moiety. (The complementary molecule is thus itself also a linking molecule having a linking moiety.) For example, biotin is a linking molecule capable of binding specifically to streptavidin. Exemplary pairs of binding partners include biotin and streptavidin, and digoxigenin and anti-digoxigenin antibodies.

B. Description of Embodiments of the Methods, Devices and Systems

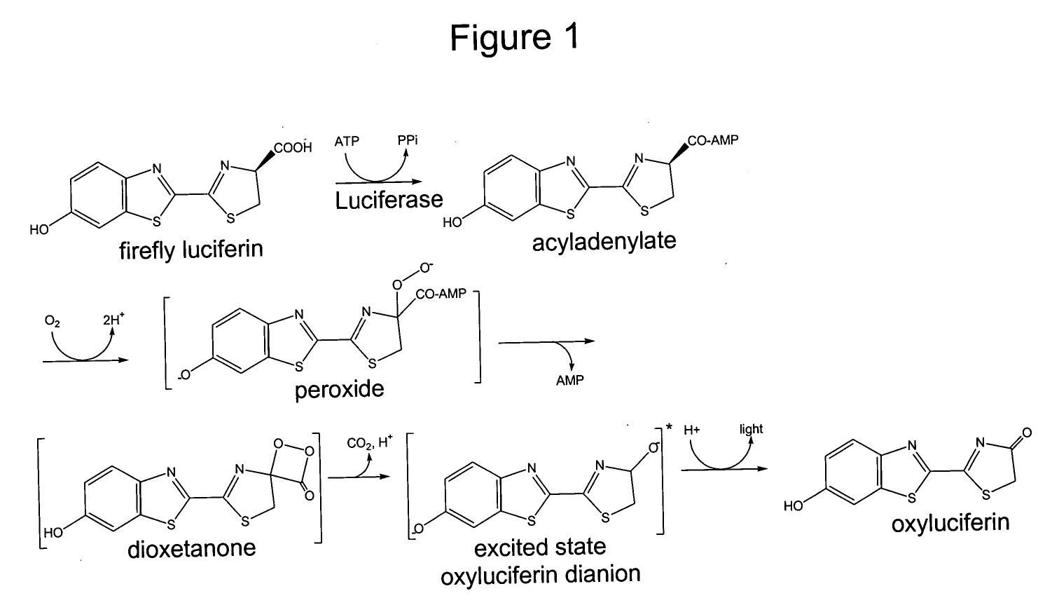

In the following, lucifierin is used as an example of a substrate that is capable of generating a useful chemiluminescent signal. However, it will be understood that lucifierin is one example of many, and that any substrate capable of generating a useful chemiluminescent signal may be used in the practice of embodiments of the invention. As illustrated in FIG. 1, firefly luciferase enzyme catalyzes the reaction of firefly luciferin to oxyluciferin. This reaction is accompanied by the emission of light, typically in the yellow-green region of the spectrum. Required cofactors include ATP and magnesium ions, which may be provided in the solution containing the luciferin (the “substrate solution”). Typically, luciferin at a concentration of between about 0.5 mM and about 5 mM is effective to provide a detectable chemiluminescent reaction. ATP is typically present in a substrate solution at a concentration of between about 1 mM and about 5 mM, preferably between about 1 mM and about 3 mM, more preferably at a concentration of about 2 mM. Magnesium ions are typically present in a substrate solution at a concentration of between about 2 mM and about 10 mM, preferably between about 4 mM and about 6 mM, more preferably at a concentration of about 5 mM.

Light emitted by the chemiluminescent reaction may be detected by a photodetector such as a luminometer, spectrophotometer, scintillation counter, a charge-coupled device (CCD) camera or sensor, a photomultiplier tube, or any suitable device or method capable of detecting light emitted by luciferase. The light detector is preferably able to discriminate between at least two wavelengths of light, so that light emitted by two different luciferases, for example, may be independently detected and discriminated. Thus, a suitable light detector will be able to detect and preferably quantify the intensity of light received at different wavelengths.

Fluorescence may be detected, and may be quantified, by any suitable means known in the art. Generally, the detection and analysis means may be any detection apparatus to provides a readout that reflect the intensity of a fluorescence signal, or that reflects the ratio of intensities of the signals generated by the first and second fluorescent indicators. Such apparatus is well known in the art, as exemplified by U.S. Pat. Nos. 4,577,109 and 4,786,886 and references such as The Photonics Design & Applications Handbook, 39th Edition (Laurin Publishing Co., Pittsfield, Mass., 1993). A detector or detector system may include, for example, optical components operationally associated with a sample holder, sample well, plate holder, closed reaction chamber, or other element for positioning the material to be inspected. Optical components may include, for example, a lens for focusing an excitation beam and for collecting the resulting fluorescence or emitted light, and a fiber optic for transmitting both the excitation beam from a light source to the lens and the fluorescent signals from the lens to a detection and analysis means. Detection means may include spectrophotometers, photomultipliers, charge-coupled detectors, and other devices capable of detecting light. For example, fluorescence may be measured with a luminescence spectrometer (e.g., a Perkin-Elmer TaqMan™ LS-50B System). A densitometer, gel scanner, automated fluorescent gel electrophoresis scanner apparatus such as the ABI Model 370, Applied Biosystems Inc., Foster City, Calif., or other instrument may be employed to detect and to measure fluorescence intensities. A photographic image may be recorded in addition to, or instead of, fluorescence detection by photodetector apparatus.

Luciferases have been isolated from several animals, purified, sequenced, and cloned. Firefly luciferase is a monomeric protein having a molecular weight of about 70 kiloDalton (kD). Wild-type luciferase enzymes typically catalyze the emission of light with maximal intensity at a wavelength of about 582 nm. Recent studies on luciferase enzymes have revealed that minor alterations in the amino acid sequence of firefly luciferase can alter the color of light emitted (see, e.g., Kajiyama and Nakano, Protein Engineering 4:691 (1991); Tatsumi et al. Anal. Biochem. 243:176-180 (1996); Mamaev et al., J. Am. Chem. Soc. 118:7243-7244 (1996); and Arslan et al., J. Am. Chem. Soc. 119:10877-10887 (1997)). For example, replacement of the serine (Ser) at position 286 of the wild-type firefly luciferase with a leucine (Leu) shifted the peak intensity wavelength to about 622 nm (appearing red to the eye). Replacing Ser286 by tyrosine (Tyr), glutamine (Gln), lysine (Lys), and by phenylalanine (Phe) also shifts the wavelength of emitted light towards red. Some luciferase variants are commercially available. For example, Kikkoman (Noda City, Japan) offers luciferase enzyme having a λₘₐₓ of 556 nm (at pH 7.8) and a luciferase enzyme having a λₘₐₓ of 618 nm at pH 7.8. These enzymes are available in biotinylated form; that is, a biotin molecule is covalently linked to these luciferase molecule. As is well-known, a biotin molecule strongly and rapidly adheres to a streptavidin molecule.

Target molecules may be tagged with luciferases by bonding the markers to the target. Such bonding may be either covalent or non-covalent, and may include antibody reactions, condensation reactions, van der Waals interactions, and hydrogen bonding to complementary structures. Nucleic acid hybridization is a fundamental process forming the basis of a wide variety of biological research techniques and clinical applications. Hybridization-based methods are useful in the detection, quantitation and/or analysis of nucleic acids. Probes useful for hybridization methods include nucleic acid species such as 2′-deoxyribonucleic acid and ribonucleic acid (i.e., DNA and RNA) structures, modified nucleotides, or nucleic acid analogs such as, for example, peptide nucleic acids (PNA) or locked nucleic acids (LNA). Antibodies bind specifically to their antigen molecules, or portions of their antigen molecules, and may be used to detect, quantify, and identify molecules such as polypeptides and proteins.

In embodiments of the methods, systems and devices, a target molecule has, or is modified to have, a linking moiety and a luciferase has, or is modified to have, a linking moiety complementary to that on the target molecule. Contact between the target molecule and the luciferase is effective to link the target with the luciferase, via the linking moieties, resulting in labeling the target molecule with the luciferase. In the presence of luciferin, ATP and Mg⁺⁺ (i.e., in a substrate solution), the luciferase catalyzes the emission of light effective to signal the presence of the linked target molecule.

Embodiments further include a probe that binds a target molecule. A probe is preferably attached to a surface, and more preferably is attached to a surface at a known or identifiable position. Target molecules present in a solution containing the probe will bind to the probe. Target molecules linked to a luciferase enzyme in a substrate solution containing probes may be detected by detection of chemiluminescent light emitted by the attached luciferase enzyme.

Where target molecules are present in a solution bathing a support surface having attached probes, the targets will adhere to the probes and so adhere to, or be held near to, the support surface. Target molecules linked to a luciferase enzyme in a substrate solution bathing a support surface having attached probes may be detected by detection of light emitted by the attached luciferase enzyme, and may be identified by determination of the position on the support surface from which the light is detected.

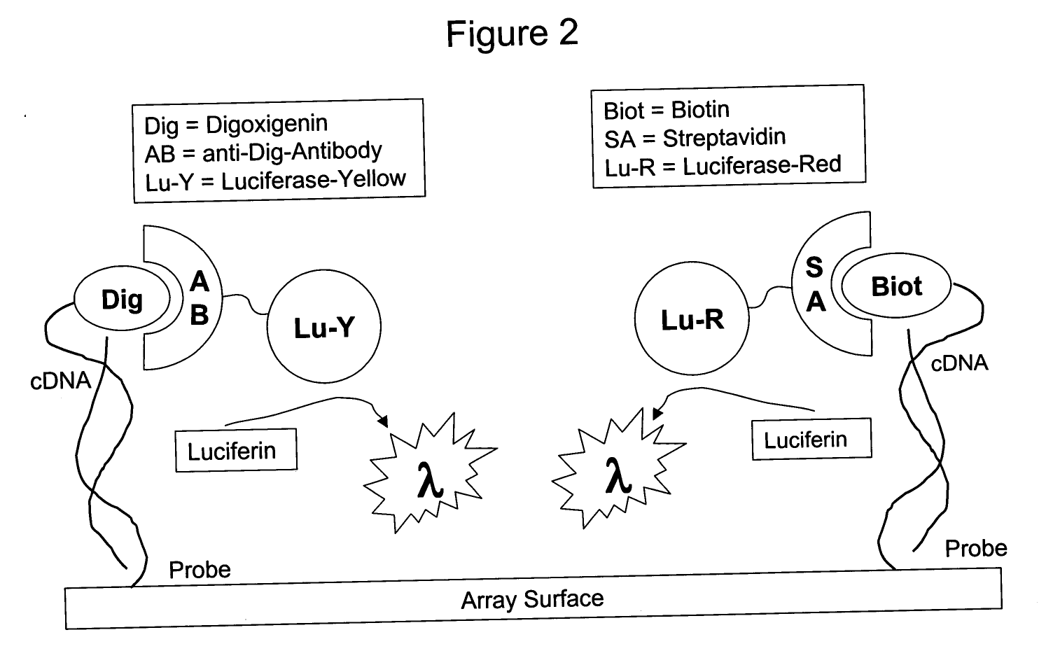

FIG. 2 is a schematic illustration of a support surface having probes attached to the surface. The probes are complementary to target molecules which have linker moieties. Also shown are luciferase enzymes having linker moieties complementary to the linker moieties of the target molecules. It will be understood that the substrate surface, target molecules, and luciferase enzymes are bathed by or present in a substrate solution. As illustrated in FIG. 2, a luciferase enzyme linked to streptavidin is utilized for recognizing and linking to a target molecule having a biotin moiety. Another embodiment illustrated in FIG. 2 utilizes luciferase enzyme linked to an antibody to digoxigenin for recognizing and linking to a target molecule having a digoxigenin moiety. However, it will be understood that other antibodies and other linker molecules for linking a luciferase enzyme to a target molecule may be used in other embodiments of the method and apparatus disclosed herein.

It will be understood that digoxigenin and biotin are examplary only, and that other labels and label pairs are also suitable for the practice of the invention. Suitable binding pairs include, but are not limited to, antigens/antibodies, including digoxigenin/antibody, dinitrophenyl (DNP)/anti-DNP, dansyl-X/anti-dansyl, fluorescein/anti-fluorescein, lucifer yellow/anti-lucifer yellow, rhodamine/anti-rhodamine; and biotin/avidin (or biotin/streptavidin). Preferred binding pairs (i.e., first and second labeling moieties) generally have high affinities for each other.

In embodiments, the smaller of the binding partners typically serves as the first labeling moiety, as steric considerations in agent:target binding may be important. Thus, preferred first labeling moieties (when second labeling moieties are used), include, but are not limited to, haptens such as biotin, etc. Biotinylation of target molecules is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. Similarly, a large number of haptenylation reagents are also known in the art.

In embodiments, multiple markers may be used. For example, a luciferase enzyme having one λₘₐₓ may be linked to streptavidin and, present in the same solution, there may be a luciferase enzyme having a second λₘₐₓ that is linked to an antibody to digoxigenin. For example, as illustrated in FIG. 2, a luciferase enzyme having a λₘₐₓ of 618 nm is linked to streptavidin and a luciferase enzyme having a λₘₐₓ of 556 nm is linked to an antibody to digoxigenin. The probe molecules are arranged on the support surface in a pattern, so that the identity of the probe molecules at any given position on the support surface is known. Detection of light emitted from a particular location on the support surface is effective to identify target molecules bound to probes at that location, since the identities of the probes are known. Detection of the λₘₐₓ of the light emitted from a location on the support surface is effective to identify the target molecules, so that multiple targets may be separately identified on a single support surface. Detection of different wavelengths of light allows detection even in a situation where both target molecules bind to the same probe or probes, since the different wavelengths of light may be discriminated and independently detected. Such detection of different wavelengths of light may occur under the same conditions and may occur at the same time.

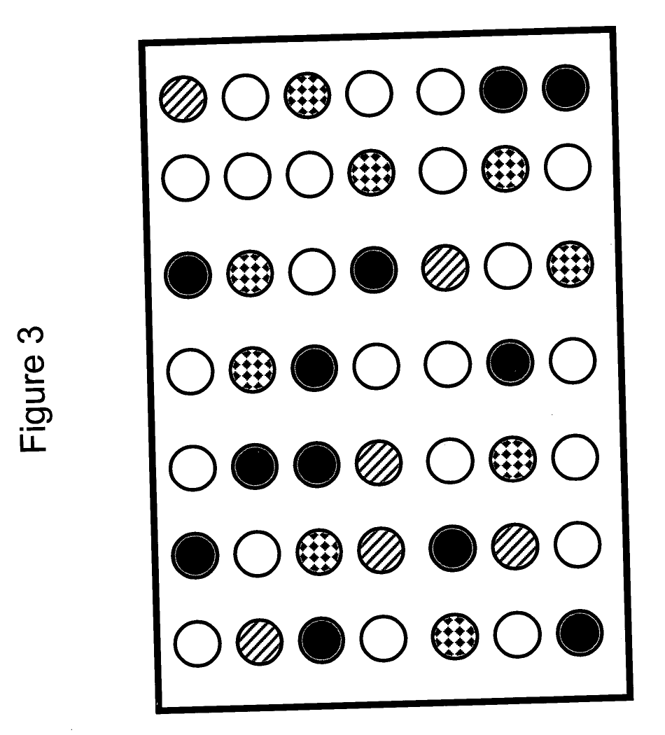

An array of probes attached to a support surface is illustrated in schematic form in FIG. 3. It will be understood that an array may include only one or a few probes, or may include probes numbered in the tens, hundreds, thousands, tens of thousands or more. The rectangle shown in FIG. 3 represents a solid support surface, such as a microscope slide, to which probes are attached. The circles arranged within the rectangle in FIG. 3 represent different locations at which different identified probe molecules are attached. The probes may be attached in arrays having any suitable pattern, such as the rectangular array illustrated. FIG. 3 represents an array to which has been applied a substrate solution and two populations of target molecules labeled with linker moieties. As indicated by the dark, striped, checked, and light circles, at some locations on the support surface, there is little or no binding of target molecules (white circles), at others one target molecule binds (striped and checked circles) and at still other locations, both target molecules bind (dark circles). Two populations of target molecules binding to such an array may be detected by light emission measurements. Such light emission measurements may be made at the same time to allow simultaneous detection of the target molecules.

In some embodiments, probes are attached to a support surface to form an “array”, “microarray”, “chip” or “biochip” as widely known in the art (see, e.g., Ausubel et al. (eds.), Current Protocols in Molecular Biology, Chapter 22, “Nucleic Acid Arrays,” John Wiley & Sons, Inc., New York [1994]; and M. Schena, (ed.), Microarray Biochip Technology, BioTechnique Books, Eaton Publishing, Natick, Mass. [2000]). In general, array formats facilitate automated analysis of large numbers of samples and/or have a large number of addressable locations, so that patterns of gene expression for a very large number of genes can be studied very rapidly.

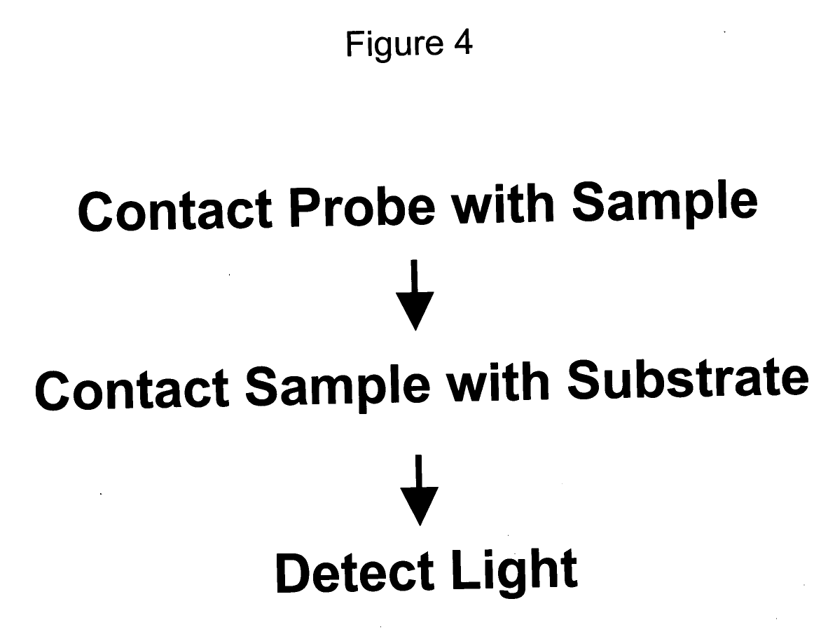

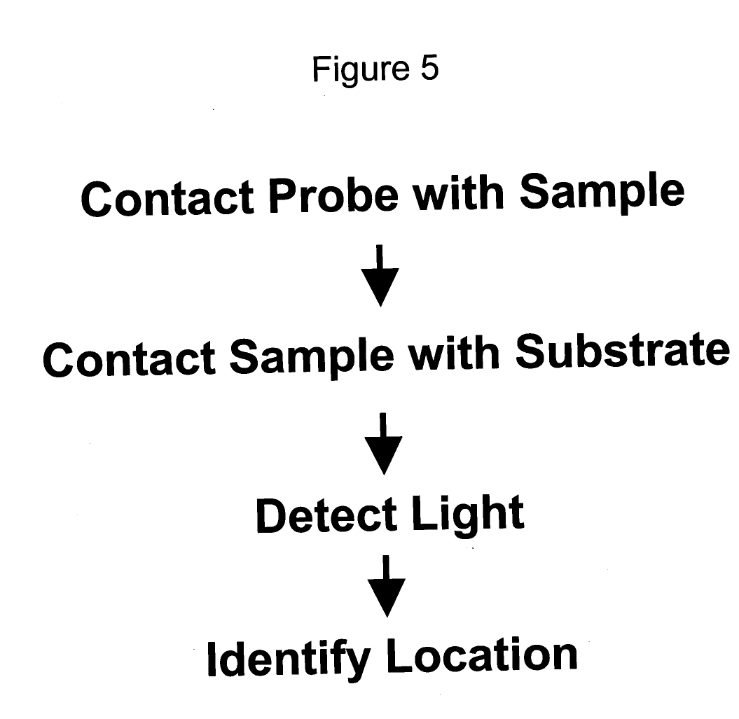

These examples illustrate some embodiments of the methods for detecting target molecules in an assay provided herein; further embodiments provide methods for identifying target molecules. In some methods, target molecules are detected and identified by detection of light emitted from luciferases bound to probe-target conjugates on a microarray. For example, a target molecule in a sample may be detected by contacting a probe with a sample including the target molecule, contacting the target with a substrate solution including labeled luciferase enzyme, effective to produce chemiluminescent light from luciferase enzyme in the substrate solution, and detecting the chemiluminescent light produced. This is illustrated in FIG. 4. A target molecule in a sample may be identified by contacting a probe attached at an identified position on a support surface with a sample including the target molecule, contacting the target with a substrate solution including a labeled luciferase enzyme, effective to produce chemiluminescent light from luciferase enzyme in the substrate solution, detecting the chemiluminescent light produced, and identifying the location on the support surface from which the chemiluminescent light was emitted effective to identify the target. This is schematically illustrated in FIG. 5.

Thus, a method for detecting target molecules in an assay includes contacting a first labeled target molecule with a first conjugate, where the first conjugate comprises a first complement conjugated with a first luciferase having a first λₘₐₓ, and said first complement has a linking moiety effective to bind said first label, and contacting a second labeled target molecule with a second conjugate, wherein said second conjugate comprises a second complement conjugated with a second luciferase having a second λₘₐₓ, the second λₘₐₓ being different than the first λₘₐₓ, and the second complement comprises a molecule effective to bind said second label. The first complement binds to the first label, and the second complement binds to the second label. Light emission from the labels may be detected to identify the target molecules. Such light emission from the first and from the second labels may be measured at the same time or at different times. Methods may further include providing a luciferase substrate, effective to induce light emission from both said first luciferase and from said second luciferase. The methods may also include providing a first target molecule labeled with a first label; providing a second target molecule labeled with a second label.

The novel methods and systems further provide a two-color target molecule assay system, comprising a first target molecule labeled with a biotin label; a second target molecule labeled with a digoxigenin label; a first conjugate comprising streptavidin conjugated with a first luciferase having a first λₘₐₓ, wherein a λₘₐₓ is defined by the wavelength of maximum intensity of light emitted by a chemiluminscent molecule, wherein said first conjugate is configured to bind to said first labeled target molecule; a second conjugate comprising an anti-digoxigenin antibody conjugated with a second luciferase having a second λₘₐₓ, wherein said second conjugate is configured to bind to said second labeled target molecule; and a luciferase substrate. In some systems, luciferin is provided in a pH-buffered substrate solution (with a pH of between about 7.0 and about 9.0) at a concentration of between about 0.5 mM and about 5 mM.

Novel labeled microarrays are also provided. For example, a microarray having features disclosed herein includes multiple probes attached to a solid support, preferably a support surface; a first target molecule labeled with a biotin label and hybridized to at least one of the probes; a first conjugate comprising streptavidin conjugated with a first luciferase having a first λₘₐₓ, the streptavidin being bound to the biotin of said first labeled target molecule; a second target molecule labeled with a digoxigenin label and hybridized to a probe; and a second conjugate including an anti-digoxigenin antibody conjugated with a second luciferase having a second λₘₐₓ, the anti-digoxigenin antibody being bound to the digoxigenin of said second labeled target molecule.

A labeled support surface may similarly include multiple probes attached to a surface of a microarray surface; a first target molecule labeled with a biotin label and hybridized to a probe; a first conjugate including streptavidin conjugated with a first luciferase having a first λₘₐₓ, the streptavidin being bound to the biotin of the first labeled target molecule; a second target molecule labeled with a digoxigenin label and hybridized to a probe; and a second conjugate including an anti-digoxigenin antibody conjugated with a second luciferase having a second λₘₐₓ, the anti-digoxigenin antibody being bound to the digoxigenin of the second labeled target molecule.

Methods may also include detecting the emitted light. Detecting the emitted light may include detecting the intensity of emitted light within each of a plurality of wavelength regions. For example, light emitted from a luciferase may be detected in a window comprising wavelengths within about 5 nm of a nominal wavelength; or, in a window comprising wavelengths within about 1 nm of a nominal wavelength. Detection of light emitted from luciferases will typically include detection of light in two or more wavlength regions, and may include a first λₘₐₓ and said second λₘₐₓ of a first and a second luciferase molecule. Typically, a nominal wavelength (around which a window is centered) will be either a first λₘₐₓ of a first luciferase or a second λₘₐₓ of a second luciferase. However, a window need not be centered on, nor need not be near, a λₘₐₓ of a luciferase. For example, windows may be centerd at wavelengths selected for desired noise characteristics, or in order to accomodate characteristics of the detection apparatus or of experimental conditions, or in order to obtain a desired separation between window wavelengths. Light emitted in two or more wavlength regions may be detected at the same time; alternatively, light emitted in two or more wavlength regions may be detected at different times.

Preferred chemiluiminescent marker molecules include luciferases, including thermostable luciferase emitting yellow light and having a λₘₐₓ of about 556 nm, and thermostable luciferase emitting red light and having a λₘₐₓ of about 618 nm. Such luciferase enzymes are produced commercially from recombinant E. coli and are available from Kikkoman Corporation (Noda City, Japan).

The substrate of the firefly luciferase enzyme, for all values of λₘₐₓ, is luciferin (see FIG. 1). The reaction providing light from firefly luciferase also requires magnesium and ATP. Thus, luciferin is typically provided in a solution also including magnesium and adenosine triphosphate (ATP), preferably in a pH buffered solution. The pH of an effective pH-buffered substrate solution is typically between about 7.5 and about 8.5. A substrate solution may include ionized magnesium (Mg⁺⁺) at a concentration of between about 2 mM and about 10 mM and ATP at a concentration of between about 1 mM and about 5 mM, preferably having a Mg⁺⁺ concentration of between about 4 mM and about 6 mM and an ATP concentration of between about 1 mM and about 3 mM.

A probe molecule may be bound to a solid support, and may be bound to a solid surface. The solid support may be a substantially flat surface. Such probe molecules and such solid surface may comprise a microarray of probe molecules arrayed on the surface, may comprise a microtitre plate having probe molecules attached to a surface in a well, or other arrangement.

The support may be a polymer, glass, or other solid-material support. For example, a probe molecule may be bound to a solid support made with glass, silica, controlled-pore-glass (CPG), reverse-phase silica, organic polymers (e.g., polystyrene, polyethylene, polypropylene, polyfluoroethylene, polyethyleneoxy, polyacrylamide, polyvinylchloride, and co-polymers and mixtures thereof), oligosaccharides, nitrocellulose, diazocellulose, dextran, agar, agarose, Sepharose®, Sephadex® Sephacryl®, cellulose, starch, nylon, latex beads, magnetic beads, paramagnetic beads, or superparamagnetic beads. A wide variety of solid supports find use with the invention, and it is not intended that the invention be limited to the use of any particular type of solid support.

In some embodiments, the surface is relatively hydrophilic, i.e., wettable surface, such as a surface having native, bound or covalently attached charged groups. The configuration of a solid support may be in the form of beads, spheres, particles, granules, a gel, or a surface. Surfaces may be planar, substantially planar, or non-planar. Solid supports may be porous or non-porous, and may have swelling or non-swelling characteristics. One such surface is a glass surface having an absorbed layer of a polycationic polymer, such as poly-1-lysine.

A microarray may be produced by placement of a plurality of probes onto a surface. There exist various methods by which these arrays can be produced. The production and use of DNA microarrays, for example, is discussed in Methods in Enzymology, Vol. 303, pages 179-205 (1999). In one aspect, presynthesized probes can be affixed to the solid support of the array using any method known in the art (e.g., UV crosslinking). Methods for the chemical attachment of probes to solid support surfaces can involve the reaction of a nucleophilic group, (e.g., an amine or thiol) of the probe to be immobilized, with an electrophilic group on the solid support surface. Alternatively, the nucleophile can be present on the support and the electrophile (e.g., activated carboxylic acid) can be present on the oligomer. In one embodiment, in the case where the probes comprise PNA, the PNA used may or may not require modification prior to the immobilization reaction because PNA possesses an amino terminus in its structure.

It is not intended that the manner in which probes are affixed to the solid support be limited in any way. In one embodiment, the probes form a support-bound array of polynucleotides or polynucleotide analogs. Detailed methods for making and using arrays comprising polymeric nucleobase structures (e.g., nucleic acid, modified nucleic acids, nucleic acid analogs, or chimeric structures) are well-known in the art and are described in many sources. See, e.g., Ausubel et al. (eds.), Current Protocols in Molecular Biology, Chapter 22, “Nucleic Acid Arrays,” John Wiley & Sons, Inc., New York [1994]; and M. Schena, (ed.), Microarray Biochip Technology, BioTechnique Books, Eaton Publishing, Natick, Mass. [2000]. Any methods for the synthesis and use of nucleic acids, modified nucleic acids and nucleic acid analogues with solid supports, and more specifically arrays, are applicable for use with the present invention.

One or more probes can be covalently linked to a surface by the reaction of a suitable functional group on the oligomer with a functional group of the surface (see, e.g., Geiger et al., PNA Array technology in molecular diagnostics, Nucleosides & Nucleotides 17(9-11):1717-1724 (1998)). This method is advantageous since the probes immobilized on the surface can be highly purified and attached using a defined chemistry, thereby possibly minimizing or eliminating non-specific interactions. Numerous types of solid supports derivatized with amino groups, carboxylic acid groups, isocyanates, isothiocyanates and malimide groups are commercially available. Probes may be synthesized on the surface in a manner suitable for deprotection but not cleavage from the synthesis support (see, e.g., Weiler et al., “Hybridization based DNA screening on peptide nucleic acid (PNA) oligomer arrays,” Nucl. Acids Res., 25(14):2792-2799 (1997)). All of the above recited methods of immobilization are not intended to be limiting in any way but are merely provided by way of illustration.

Alternatively, probe molecules may be placed at identified locations on a surface by spotting (see, e.g, U.S. Pat. No. 5,807,522 to Brown et al.) or a probe array can be synthesized by ligating two or more probes directly on the solid support using linker chemistry for linking the probe to a surface by a chemical reaction. Alternatively, an array of polynculeotides or polypeptides may be synthesized at locations on a substrate using photolithographic methods (see, e.g., U.S. Pat. No. 5,800,992 to Fodor et al.).

In some exemplary embodiments, arrays or microarrays may have probe region densities of greater than 100 discrete features or regions per cm², 300 per cm², 1000 per cm², 3000 per cm², 10,000 per cm², 100,000 per cm², or 10⁶ per cm². In some embodiments, the number of different probes in each array may be equal to or greater than 10, 20, 50, 100, 200, 500, 1000, 3000, 10,000, 30,000, 100,000, or 300,000, for example.

Because the location and sequence of each support bound oligomer is known, arrays can be used to simultaneously detect, identify and/or quantitate the presence or amount of one or more target sequences in a sample. For example, a target sequence can be captured by a probe on the array surface having the complementary nucleotide sequence and then the complex containing the target sequence can be detected. Since the sequence of the probe is known at each location on the surface of the array, the sequence of target sequence(s) can be directly detected, identified and/or quantitated by determining the location of a detectable signal generated on the array. Thus, arrays are useful in diagnostic applications or in screening compounds, e.g., during development of therapeutic compounds as well as for detecting the presence of, and identifying, nucleotide sequences in a sample. Similarly, where the array is an array of antibodies having known affinities (e.g., directed against known epitopes), target polypeptides and proteins may be detected and identified.

The compositions and methods of the present invention find use in a variety of applications, some, but not all, of which are discussed in the following.

Analysis of Gene Expression

The expression of target genes may be detected with methods and systems disclosed herein. For example, a cDNA made from an mRNA obtained from a cell of interest may be prepared having a linker moiety such as biotin, as illustrated in FIG. 2, and contacted with an array having a plurality of probes complementary to some or all gene sequences that might be relevant to the cell of interest. Supply of luciferase linked to a complementary linker moiety (e.g., streptavidin), luciferin and cofactors is effective to produce a chemiluminescent signal from the luciferase and to detect and identify the cDNA. In preferred methods, two cDNAs having two different linker moieties (e.g., biotin and digoxigenin) are contacted with an array having multiple probes, a substrate solution including two different luciferases having different λₘₐₓs and attached to different linker moieties (e.g., streptavidin and anti-digoxigenin antibody) applied (the cDNAs may be applied to the array in, or along with, the substrate solution), and resulting chemiluminescent signals from the luciferases used to detect and to identify the cDNAs.

Gene expression may be analyzed by detection of target gene or other nucleobase sequences in a sample indicative of gene expression, such as a cDNA derived from mRNA obtained from a cell of interest. Such gene expression analysis may be performed on similar cells under different conditions or from cells during different parts of the cell cycle (see, for example, DeRisi et al., Science 278:680-686 (1997)). Comparison of the results of such gene expression analysis may be used to determine what gene activity is altered under the different conditions or during the different parts of the cell cycle. Similarly, comparison between normal cells and cancerous cells may indicate differences in gene expression between the normal and the cancerous conditions. Thus, for example, where cDNAs are obtained from normal and cancerous cells, comparison of the hybridization between such cDNAs and probes on an array may be used to determine differences in gene expression between normal and cancerous cells.

Probe-target conjugates may be formed by hybridization between a probe and a target. For example, where a target molecule includes a polynucleotide sequence, a probe having a complementary polynucleotide (or polynucleotide analog) sequence may bind to the target by hybridization. A wide variety of sources are available that describe hybridization conditions for particular application; see, e.g., Ausubel et al. (eds.), Current Protocols in Molecular Biology, Chapter 22, “Nucleic Acid Arrays,” John Wiley & Sons, Inc., New York [1994]; and M. Schena, (ed.), Microarray Biochip Technology, BioTechnique Books, Eaton Publishing, Natick, Mass. [2000].

The specificity of hybridization between probe and target may depend upon any of several factors. Antibody binding to an antigen, or epitope on a complementary antibody target, may be affected by pH, salt concentration, temperature, and other factors. The specificity of the hybridization between nucleobases may also be affected by these, and other factors, and the specificity may be determined by manipulation of these factors.

Preferably, such hybridization reactions take place in high throughput formats, as known in the art. Generally, the high throughput hybridization formats use a probe that is affixed to a solid support. The solid support can be any composition and configuration, and includes organic and inorganic supports, and can comprise beads, spheres, particles, granules, planar or non-planar surfaces, and/or in the form of wells, dishes, plates, slides, wafers or any other kind of support. In some embodiments, the structure and configuration of the solid support is designed to facilitate robotic automation technology. The steps of detecting, measuring and/or quantitating can also be done using automated technology.

Multiplex Analysis

In certain embodiments, provided herein are methods and systems for use in multiplex hybridization assays. In a multiplex assay, numerous conditions of interest are simultaneously or sequentially examined. Multiplex analysis relies on the ability to sort sample components or the data associated therewith, during or after the assay is completed. In performing a multiplex assay, one or more distinct independently detectable moieties can be used to label two or more different targets that are to be used simultaneously in an assay. As used herein, “independently detectable” means that it is possible to determine one label independently of, and in the presence of, at least one other additional label. For example, a luciferase enzyme having a λₘₐₓ of 556 nm is independently detectable in the presence of a luciferase enzyme having a λₘₐₓ of 618 nm. The binding of two targets to probes on an array may be determined independently by detection and quantitization of chemiluminescent light emission from different locations on the array, the light measurements being obtained at two different wavelengths (e.g., by the use of filters to pass only a desired wavelength, or by the use of a prism or other refractive device to direct light of a desired wavelength to a detector without directing other wavelengths to it). The assays of this invention can, for example, be used to simultaneously or sequentially detect the presence, absence, number, position or identity of two target sequences in the same sample in the same assay.

Detection/Identification of Biological Organisms

Methods and systems disclosed herein find use in the detection, identification and/or enumeration of biological organisms, and especially, pathogens. Such organisms can include viruses, bacteria and eucarya in food, beverages, water, pharmaceutical products, personal care products, dairy products or in samples of plant, animal, human or environmental origin, including in clinical specimens, equipment, fixtures or products used to treat humans or animals as well as in clinical samples and clinical environments. One or more target molecules may be obtained from a specimen or derived from a specimen (e.g., a target molecule obtained using PCR to amplify a particular nucleic acid sequence). The target molecules are preferably unique to, or indicative of, the source organism or specimen. The target molecules are provided with a linker moiety (e.g., biotin or digoxigenin) and contacted with a probe (e.g., a surface having an array of probes molecules attached), a substrate solution including luciferase enzymes linked to linker moieties complementary to the linker of the target and luciferin, ATP and Mg⁺⁺, and resulting light detected and localized to a portion or portion of the substrate to identify the target or targets.

The compositions, methods, kits, libraries and arrays disclosed herein are particularly useful in areas such as expression analysis, single nucleotide polymorphism (SNP) analysis, genetic analysis of humans, animals, fungi, yeast, viruses, and plants (including genetically modified organisms), therapy monitoring, pharmacogenomics, pharmacogenetics, epigenomics, and high throughput screening operations.

All publications, patents and published patent applications mentioned in the above specification are herein incorporated by reference in their entirety. Various modifications and variations of the described compositions and methods of the invention will be apparent to those skilled in the art without departing from the scope and spirit of the invention. Although the methods, apparatus and systems have been described in connection with various specific embodiments, it should be understood that the disclosed methods, apparatus and systems should not be unduly limited to such specific embodiments. Indeed, various modifications of the described modes for making and using the methods, apparatus and systems disclosed herein which are obvious to those skilled in the art are intended to be within the scope of the following claims.

What is claimed is:

1. A method for detecting tagged target molecules present in a sample, comprising:
providing a plurality of probe molecules bound to a solid support, said probe molecules comprising probe molecules complementary to target molecules;
contacting a first target molecule having a first tag with a first anti-tag complex, wherein said first anti-tag complex comprises a first anti-tag conjugated with a first luciferase having a first λₘₐₓ, wherein a λₘₐₓ is defined by the wavelength of maximum intensity of light emitted by a chemiluminscent molecule and said first anti-tag binds to said first tag;
contacting a second target molecule having a second tag with a second anti-tag complex, wherein said second anti-tag complex comprises a second anti-tag conjugated with a second luciferase having a second λₘₐₓ, wherein said second λₘₐₓ is different than said first λₘₐₓ, and said second anti-tag binds to said second tag;
providing a luciferase substrate, effective to induce light emission catalyzed at the same time by both said first luciferase and by said second luciferase; and
determining a location of said first luciferase and of said second luciferase by detecting emitted light effective to identify a tagged target molecule.

2. The method for detecting tagged target molecules present in a sample of claim 1, wherein said detecting emitted light comprises detecting emitted light from said first luciferase and of said second luciferase at the same time.

3. The method for detecting tagged target molecules present in a sample of claim 1, further comprising providing a first target molecule having a first tag and providing a second target molecule having a second tag.

4. The method of claim 1, wherein said first λₘₐₓ is less than about 600 nm, and wherein said second λₘₐₓ is greater than about 600 nM.

5. The method of claim 1, wherein said first λₘₐₓ is between about 540 nm and about 590 nm, and wherein said second λₘₐₓ is between about 600 nM and about 650 nm.

6. The method of claim 1, wherein said first λₘₐₓ is between about 550 nm and about 560 nm, and wherein said second λₘₐₓ is between about 610 nM and about 620 nm.

7. The method of claim 1, wherein detecting emitted light comprises detecting the intensity of emitted light in a wavelength region including a λₘₐₓ of a luciferase, wherein a wavelength region comprises a range of wavelengths near to a nominal wavelength.

8. The method of claim 1, wherein said detecting comprises detecting the intensity of emitted light within each of a plurality of wavelength regions.

9. The method of claim 8, wherein said plurality of wavelength regions comprises a wavelength region including a wavelength selected from said first λₘₐₓ and said second λₘₐₓ.

10. The method of claim 8, wherein said plurality of wavelength regions comprises a first wavelength region including said first λₘₐₓ and a second wavelength region including said second λₘₐₓ.

11. The method of claim 8, wherein said wavelength regions comprise a wavelength region including the wavelength 556 nm and and a wavelength region including the wavelength 618 nm.

12. The method of claim 2, further comprising providing a first target molecule having a first tag and providing a second target molecule having a second tag.

13. The method of claim 2, wherein said first λₘₐₓ is less than about 600 nm, and wherein said second λₘₐₓ is greater than about 600 nM.

14. The method of claim 2, wherein said first λₘₐₓ is between about 540 nm and about 590 nm, and wherein said second λₘₐₓ is between about 600 nM and about 650 nm.

15. The method of claim 1, wherein said solid support comprises a material selected from glass, silica, controlled-pore-glass (CPG), reverse-phase silica, organic polymers, polystyrene, polyethylene, polypropylene, polyfluoroethylene, polyethyleneoxy, polyacrylamide, polyvinylchloride, and co-polymers and mixtures thereof, oligosaccharides, nitrocellulose, diazocellulose, dextran, agar, agarose, Sepharose®, Sephadex® Sephacryl®, cellulose, starch, nylon, latex beads, magnetic beads, paramagnetic beads, and superparamagnetic beads.

16. The method of claim 1, wherein said solid support comprises a substantially flat surface.

17. The method of claim 16, wherein said probe molecules comprise an array of probe molecules on said substantially flat surface.

18. The method of claim 17, wherein said substantially flat surface comprises a material selected from glass, silica, controlled-pore-glass (CPG), reverse-phase silica, polystyrene, polyethylene, polypropylene, polyfluoroethylene, polyethyleneoxy, polyacrylamide, polyvinylchloride, organic polymers, and co-polymers and mixtures thereof.

19. The method of claim 17, wherein said probe molecules and said substantially flat surface comprise a microarray.

20. The method of claim 17, wherein said probe molecules and said substantially flat surface comprise a microtitre plate having wells with substantially flat well surfaces, and wherein said probe molecules are attached to at least one substantially flat well surface.

21. The method of claim 1, wherein providing said luciferin comprises providing between about 0.5 mM and about 5 mM luciferin in a substrate solution comprising ionized magnesium (Mg⁺⁺) at a concentration of between about 2 mM and about 10 mM and ATP at a concentration of between about 1 mM and about 5 mM at a pH of between about 7.5 and about 8.5.

22. The method of claim 21, wherein said substrate solution comprises ionized magnesium (Mg⁺⁺) at a concentration of between about 4 mM and about 6 mM and ATP at a concentration of between about 1 mM and about 3 mM.

23. A method for labeling two target molecules present in a sample, comprising:
contacting a first tagged target molecule with a first anti-tag complex comprising streptavidin coupled with a first luciferase having a first λₘₐₓ, wherein said first tagged target molecule comprises a target molecule coupled with a biotin label, and wherein said streptavidin binds to said biotin; and
contacting a second tagged target molecule with a second anti-tag complex comprising an anti-digoxigenin antibody coupled with a second luciferase having a second λₘₐₓ, wherein said second tagged target molecule comprises a target molecule coupled to digoxigenin and wherein said anti-digoxigenin antibody binds to said digoxigenin,
whereby said first target molecule is labeled with a first luciferase and said second target molecule is labeled with a second luciferase.

24. The method of claim 23, further comprising providing said first tagged target molecule coupled with a biotin label and further comprising providing said second target molecule labeled with a digoxigenin label.

25. The method of claim 23, wherein said first λₘₐₓ comprises a wavelength of about 556 nm and said second λₘₐₓ comprises a wavelength of about 618 nm.

26. A two-color target molecule assay system, comprising:
a first target molecule labeled with a biotin label;
a second target molecule labeled with a digoxigenin label;
a first conjugate comprising streptavidin conjugated with a first luciferase having a first λₘₐₓ, wherein a λₘₐₓ is defined by the wavelength of maximum intensity of light emitted by a chemiluminscent molecule, wherein said first conjugate is configured to bind to said first labeled target molecule;
a second conjugate comprising an anti-digoxigenin antibody conjugated with a second luciferase having a second λₘₐₓ, wherein said second conjugate is configured to bind to said second labeled target molecule; and
a luciferase substrate.

27. The two-color target molecule labeling system of claim 26, wherein said first λₘₐₓ comprises a wavelength of less than about 600 nm and said second λₘₐₓ comprises a wavelength of greater than about 600 nm.

28. The two-color target molecule labeling system of claim 26, wherein said first λₘₐₓ comprises a wavelength of between about 550 nm and about 590 nm, and said second λₘₐₓ comprises a wavelength of between about 600 nm and about 640 nm.

29. The two-color target molecule labeling system of claim 26, wherein said first λₘₐₓ comprises a wavelength of between about 550 nm and about 560 nm, and said second λₘₐₓ comprises a wavelength of between about 610 nm and about 620 nm.

30. The two-color target molecule labeling system of claim 26, wherein said first λₘₐₓ comprises a wavelength of about 556 nm and said second λₘₐₓ comprises a wavelength of about 618 nm.

31. The two-color target molecule labeling system of claim 26, wherein said luciferin is provided in a pH-buffered substrate solution at a concentration of between about 0.5 mM and about 5 mM, said substrate solution having a pH of between about 7.0 and about 9.0.

32. The two-color target molecule labeling system of claim 31, wherein said substrate solution further comprises magnesium and adenosine triphosphate (ATP).

33. The two-color target molecule labeling system of claim 31, wherein the pH of said pH-buffered substrate solution is between about 7.5 and about 8.5.

34. The two-color target molecule labeling system of claim 32, wherein said substrate solution comprises ionized magnesium (Mg⁺⁺) at a concentration of between about 2 mM and about 10 mM and ATP at a concentration of between about 1 mM and about 5 mM.

35. The two-color target molecule labeling system of claim 32, wherein said substrate solution comprises ionized magnesium (Mg⁺⁺) at a concentration of between about 4 mM and about 6 mM and ATP at a concentration of between about 1 mM and about 3 mM.

36. A labeled microarray, comprising:
a plurality of probes attached to a solid surface;
a first target molecule labeled with a biotin label and hybridized to at least one of said probes;
a first conjugate comprising streptavidin conjugated with a first luciferase having a first λₘₐₓ, wherein a λₘₐₓ is defined by the wavelength of maximum intensity of light emitted by a chemiluminscent molecule, wherein said streptavidin is bound to said biotin of said first labeled target molecule;
a second target molecule labeled with a digoxigenin label and hybridized to at least one of said probes; and
a second conjugate comprising an anti-digoxigenin antibody conjugated with a second luciferase having a second λₘₐₓ, wherein said anti-digoxigenin antibody is bound to said digoxigenin of said second labeled target molecule.

37. The labeled microarray of claim 36, wherein said first λₘₐₓ comprises a wavelength of less than about 600 nm and said second λₘₐₓ comprises a wavelength of greater than about 600 nm.

38. The labeled microarray of claim 36, wherein said first λₘₐₓ comprises a wavelength of between about 550 nm and about 590 nm, and said second λₘₐₓ comprises a wavelength of between about 600 nm and about 640 nm.

39. The labeled microarray of claim 36, wherein said first λₘₐₓ comprises a wavelength of between about 550 nm and about 560 nm, and said second λₘₐₓ comprises a wavelength of between about 610 nm and about 620 nm.

40. The labeled microarray of claim 36, wherein said first λₘₐₓ comprises a wavelength of about 556 nm and said second λₘₐₓ comprises a wavelength of about 618 nm.

41. A labeled microtitre plate having wells with well surfaces, comprising:
a plurality of probes attached to a well surface;
a first target molecule labeled with a biotin label and hybridized to at least one of said probes;
a first conjugate comprising streptavidin conjugated with a first luciferase having a first λₘₐₓ, wherein a λₘₐₓ is defined by the wavelength of maximum intensity of light emitted by a chemiluminscent molecule, wherein said streptavidin is bound to said biotin of said first labeled target molecule;
a second target molecule labeled with a digoxigenin label and hybridized to at least one of said probes; and
a second conjugate comprising an anti-digoxigenin antibody conjugated with a second luciferase having a second λₘₐₓ, wherein said anti-digoxigenin antibody is bound to said digoxigenin of said second labeled target molecule.

42. The labeled microtitre plate of claim 41, wherein said first λₘₐₓ comprises a wavelength of less than about 600 nm and said second λₘₐₓ comprises a wavelength of greater than about 600 nm.

43. The labeled microtitre plate of claim 41, wherein said first λₘₐₓ comprises a wavelength of between about 550 nm and about 590 nm, and said second λₘₐₓ comprises a wavelength of between about 600 nm and about 640 nm.

44. The labeled microtitre plate of claim 41, wherein said first λₘₐₓ comprises a wavelength of between about 550 nm and about 560 nm, and said second λₘₐₓ comprises a wavelength of between about 610 nm and about 620 nm.

45. The labeled microtitre plate of claim 41, wherein said first λₘₐₓ comprises a wavelength of about 556 nm and said second λₘₐₓ comprises a wavelength of about 618 nm.

46. A labeled support surface, comprising:
a plurality of probes attached to a said surface;
a first target molecule labeled with a biotin label and hybridized to at least one of said probes;
a first conjugate comprising streptavidin conjugated with a first luciferase having a first λₘₐₓ, wherein a λₘₐₓ is defined by the wavelength of maximum intensity of light emitted by a chemiluminscent molecule, wherein said streptavidin is bound to said biotin of said first labeled target molecule;
a second target molecule labeled with a digoxigenin label and hybridized to at least one of said probes; and
a second conjugate comprising an anti-digoxigenin antibody conjugated with a second luciferase having a second λₘₐₓ, wherein said anti-digoxigenin antibody is bound to said digoxigenin of said second labeled target molecule.
